(19) United States
(12) Patent Application Publication
(10) Pub. No.: US 2024/0282444 A1
(43) Pub. Date: Aug. 22, 2024

(54) CONFIGURATOR FOR REMOTE MANAGEMENT OF MEDICAL DEVICES

(71) Applicant: Welch Allyn, Inc., Skaneateles Falls, NY (US)

(72) Inventors: Steven Morrow, Vestal, NY (US); Puspita Banerjee, Chesterfield, MO (US)

(21) Appl. No.: 18/435,409

(22) Filed: Feb. 7, 2024

Related U.S. Application Data
(60) Provisional application No. 63/486,293, filed on Feb. 22, 2023.

(51) Int. Cl. G16H 40/67

(52) U.S. Cl. CPC G16H 40/67

(57) ABSTRACT

An apparatus for configuring a remote management system that is used for remotely managing a plurality of medical device assets. The apparatus displays a user interface including a first plurality of selectors for enabling device component information in the remote management system, and a second plurality of selectors for enabling functionalities in the remote management system. The apparatus receives from the user interface one or more selections of the first and second pluralities of selectors for a medical device asset. The apparatus configures the remote management system to remotely manage the medical device asset based on the one or more selections of the first and second pluralities of selectors for the medical device asset.

### BACKGROUND

Medical devices collect, monitor, and display various aspects associated with a patient's physiology. Typically, medical devices need to be serviced at periodic intervals. For example, sensors, probes, and other components on medical devices may have a defined useful life (e.g., 6000 usage cycles) before those components must be replaced. Likewise, the software and firmware running on medical devices may need to be periodically updated.

Systems have been developed to remotely manage medical devices, such as to monitor usage and provide software and firmware updates. Due to significant differences between diverse types of medical devices, such systems require extensive customization to support a variety of aspects between the diverse types of medical devices.

### SUMMARY

In general terms, the present disclosure relates to a configurator for a remote management system that remotely manages a plurality of medical device assets. Various aspects are described in this disclosure, which include, but are not limited to, the following aspects.

One aspect relates to an apparatus for configuring a remote management system that is used for remotely managing a plurality of medical device assets, the apparatus comprising: at least one processing device; and a memory device storing instructions which, when executed by the at least one processing device, cause the at least one processing device to: display a user interface including: a first plurality of selectors for enabling device component information in the remote management system; and a second plurality of selectors for enabling functionalities in the remote management system; receive from the user interface one or more selections of the first and second pluralities of selectors for a medical device asset; and configure the remote management system to remotely manage the medical device asset based on the one or more selections of the first and second pluralities of selectors for the medical device asset.

Another aspect relates to a method of configuring a remote management system that remotely manages a plurality of medical device assets, the method comprising: displaying a user interface including: a first plurality of selectors for enabling device component information in the remote management system; and a second plurality of selectors for enabling functionalities in the remote management system; receiving from the user interface one or more selections of the first and second pluralities of selectors for a medical device asset; and configuring the remote management system to remotely manage the medical device asset based on the one or more selections of the first and second pluralities of selectors for the medical device asset.

Another aspect relates to a non-transitory computer-readable data storage medium comprising instructions that, when executed, cause at least one computing device to: display a user interface including: a first plurality of selectors for enabling device component information in a remote management system; and a second plurality of selectors for enabling functionalities in the remote management system; receive from the user interface selections of the first and second pluralities of selectors for a medical device asset; and configure the remote management system to remotely manage the medical device asset based on the selections of the first and second pluralities of selectors for a medical device asset.

### DESCRIPTION OF THE FIGURES

The following drawing figures, which form a part of this application, are illustrative of the described technology and are not meant to limit the scope of the disclosure in any manner.

FIG. 1 schematically illustrates an example of a remote management system that can be used to remotely manage a plurality of medical device asset types.

FIG. 2 schematically illustrates an example of a configurator for configuring the remote management system of FIG. 1.

FIG. 3 schematically illustrates an example of a method of configuring the remote management system of FIG. 1 using the configurator.

FIG. 4 shows an example of a user interface generated by the configurator for creating a new medical device asset in accordance with an operation of the method of FIG. 3.

FIG. 5 shows another example of a user interface generated by the configurator for editing an existing medical device asset in accordance with an operation of the method of FIG. 3.

FIG. 6 shows an example of a user interface generated by the configurator that displays a first plurality of selectors for a new or existing medical device asset in accordance with an operation of the method of FIG. 3.

FIG. 7 shows an example of a user interface generated by the configurator that displays a second plurality of selectors for the new or existing medical device asset in accordance with an operation of the method of FIG. 3.

FIG. 8 shows a user interface generated by the configurator that illustrates in more detail configuring component information details and tracking based on a selection of the second plurality of selectors included in the user interface of FIG. 7.

FIG. 9 shows an example of a user interface generated by the configurator that includes a third plurality of selectors for customizing reports in the remote management system of FIG. 1 for a new or existing medical device asset.

FIG. 10 shows another example of a user interface generated by the configurator that includes the third plurality of selectors for configuring one or more report types in the remote management system of FIG. 1 for a new or existing medical device asset.

FIG. 11 shows an example of a user interface displayed by the remote management system of FIG. 1 once configured by the configurator in accordance with the operations of the method of FIG. 3.

FIG. 12 shows an example of a user interface displayed by the remote management system of FIG. 1 after selection of a medical device asset shown in the user interface of FIG. 11.

FIG. 13 shows another example of a user interface displayed by the remote management system of FIG. 1 after a selection of another medical device asset shown in the user interface of FIG. 11.

FIG. 14 shows another example of a user interface displayed by the remote management system of FIG. 1 after selection of a locate asset icon included in the user interface of FIG. 13.

FIG. 15 shows another example of a user interface generated on the configurator for configuring the remote management system of FIG. 1.

FIG. 16 shows another example of a user interface generated on the configurator for configuring the remote management system of FIG. 1.

FIG. 17 schematically illustrates an example of computing hardware of the configurator of FIG. 2 for implementing the aspects of the present disclosure.

### DETAILED DESCRIPTION

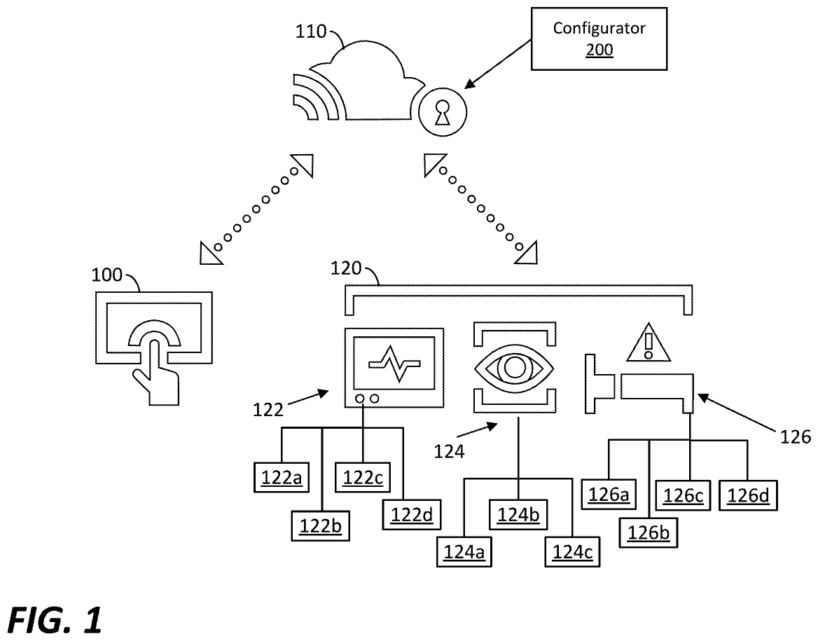

FIG. 1 schematically illustrates an example of a remote management system 100 that can be used to remotely manage a plurality of medical device asset types 120. The remote management system 100 communicates with an internet of things network 110, and the plurality of medical device asset types 120 are also communicatively connected with the internet of things network 110. In some examples, the remote management system 100 includes SmartCare Remote Management software available from Hill-Rom®, Inc. of Batesville, Ind.

A configurator 200 is also communicatively connected to the internet of things network 110. The configurator 200 is a scalable, turnkey configuration tool which configures the remote management system 100 with ala-carte features and functionalities per medical device asset type. The configurator 200 configures the remote management system 100 to enable features available in the remote management system 100 for new medical device asset types. Also, the configurator 200 configures the remote management system 100 to easily expand newly added features to existing medical device asset types. The configurator 200 can be implemented on an apparatus such as a workstation computer.

The configurator 200 significantly reduces manual work such as software coding, which is typically required to enable features in the remote management system 100 to be available for existing and new medical device asset types. The configurator 200 can significantly reduce resources such as software work product and time for configuring the features in the remote management system 100 for availability to existing and new medical device asset types.

The plurality of medical device asset types 120 can each include various medical device assets produced and sold by a medical device manufacturer. For example, the plurality of medical device asset types 120 can include a first medical device asset type 122 that represents a category of assets such as vital signs monitoring devices. Examples of the assets under the first medical device asset type 122 can include Connex® Spot Monitors 122a, Connex® Integrated Wall Systems 122b, Connex® Vital Signs Monitors 122c, and EarlySense® Vitals Surveillance Systems 122d, each available from Hill-Rom®, Inc. of Batesville, Ind. Additional assets within the first medical device asset type 122 are possible, and new assets may be added in the future. The assets in the first medical device asset type 122 are sold by the medical device manufacturer in different geographical locations for use by different customers.

As further shown in FIG. 1, the plurality of medical device asset types 120 can further include a second medical device asset type 124 which represents a category of assets such as eye imaging devices. Illustrative examples of the second medical device asset type 124 can include Retina Vue® 700 Imagers 124a, Retina Vue® 100 Imagers 124b, and Spot Vision Screeners 124c, each available from Hill-Rom®, Inc. of Batesville, Ind. Additional assets in the second medical device asset type 124 are possible, and new assets may be added in the future. The assets in the second medical device asset type 124 can be sold by the medical device manufacturer in different geographical locations for use by different customers.

The plurality of medical device asset types 120 can also include a third medical device asset type 126 that represents a category of assets such as patient support systems. Examples of the third medical device asset type 126 can include Accella® Smart Bed 126a, Progressa® Smart Beds 126b, VersaCare® Med-Surg Beds 126c, and Advanta® Med Surge Beds 126d, each available from Hill-Rom®, Inc. of Batesville, Ind. Additional assets within the third medical device asset type 126 are possible, and new assets may be added in the future. The assets in the third medical device asset type 126 can be sold by the medical device manufacturer in different geographical locations for use by different customers.

The plurality of medical device asset types 120 can include additional medical device asset types such that the first, second, and third medical device asset types are provided by way of illustrative example. The configurator 200 can be used to configure the features available in the remote management system 100 for remote management of each existing asset within an asset type of the plurality of medical device asset types 120, and for remotely managing new assets added to any one of the plurality of medical device asset types 120.

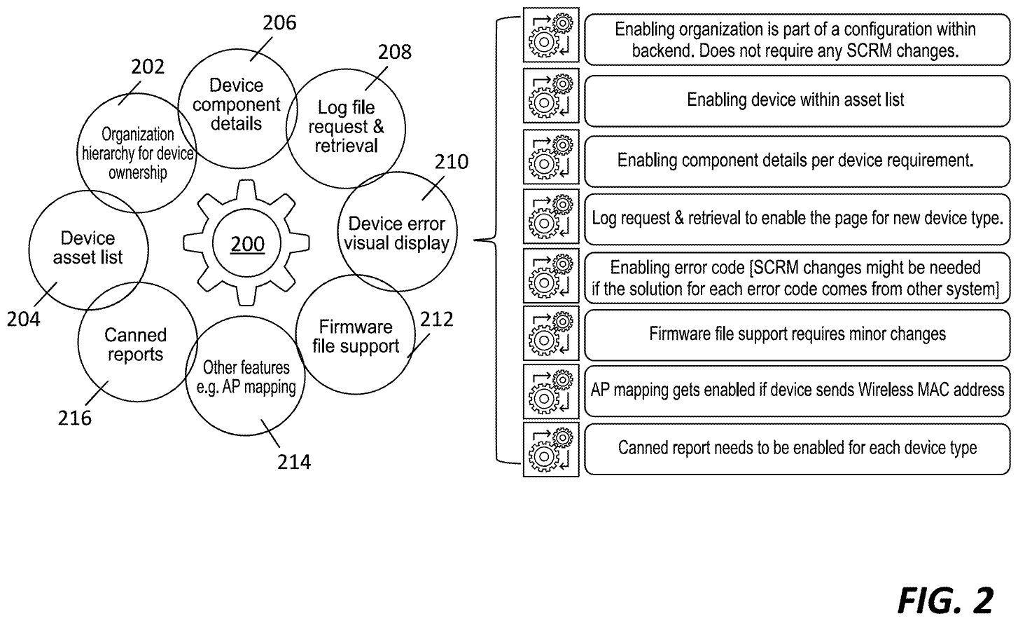

FIG. 2 schematically illustrates an example of the configurator 200 for configuring the remote management system 100. As shown in FIG. 2, the configurator 200 can be used to enable and/or disable a plurality of features in the remote management system 100 for existing and new medical device assets. The configurator 200 provides a simplified and flexible platform for a user, such as an administrator of the medical device manufacturer, to configure the remote management system 100 to operate a variety of medical device assets within the same medical device asset type, or within different medical device asset types.

As shown in FIG. 2, an administrator can manage the creation and maintenance of each medical device asset within the remote management system 100 to enable one or more features including, without limitation: a first feature 202 for enabling organization hierarchy for ownership of the medical device asset within the remote management system 100; a second feature 204 for displaying the medical device asset within an asset list maintained by the remote management system 100; a third feature 206 for enabling component information details and tracking for the medical device asset within the remote management system 100; a fourth feature 208 for enabling log request and retrieval for the medical device asset in the remote management system 100; a fifth feature 210 for enabling visual display of error codes associated with the medical device asset within the remote management system 100; a sixth feature 212 for enabling firmware file support for the medical device asset within the remote management system 100; a seventh feature 214 for enabling access point mapping for the medical device asset within the remote management system 100; and an eighth feature 216 for enabling canned reports for the medical device asset within the remote management system 100.

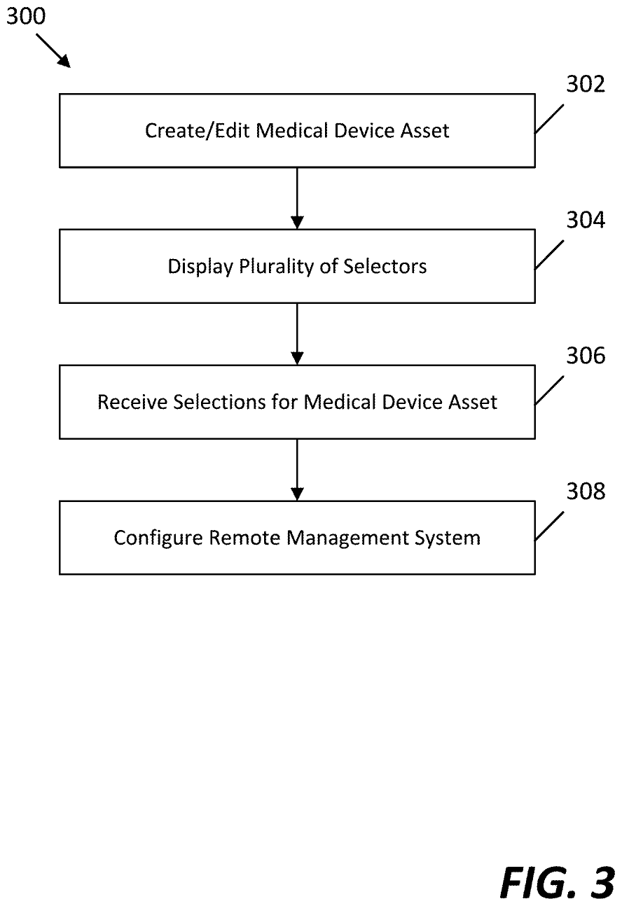

FIG. 3 schematically illustrates an example of a method 300 of configuring the remote management system 100 for remote management of an existing or new medical device asset within any of the plurality of medical device asset types 120 described above. The operations in the method 300 can be performed through use of the configurator 200.

The method 300 includes an operation 302 of creating a new medical device asset or editing an existing medical device asset using the configurator 200. Operation 302 can include entering a name for the new medical device asset, an optional abbreviated or shortened name for the new medical device asset, and an asset type for the new medical device asset. Operation 302 can further include adding an image of the new medical device asset.

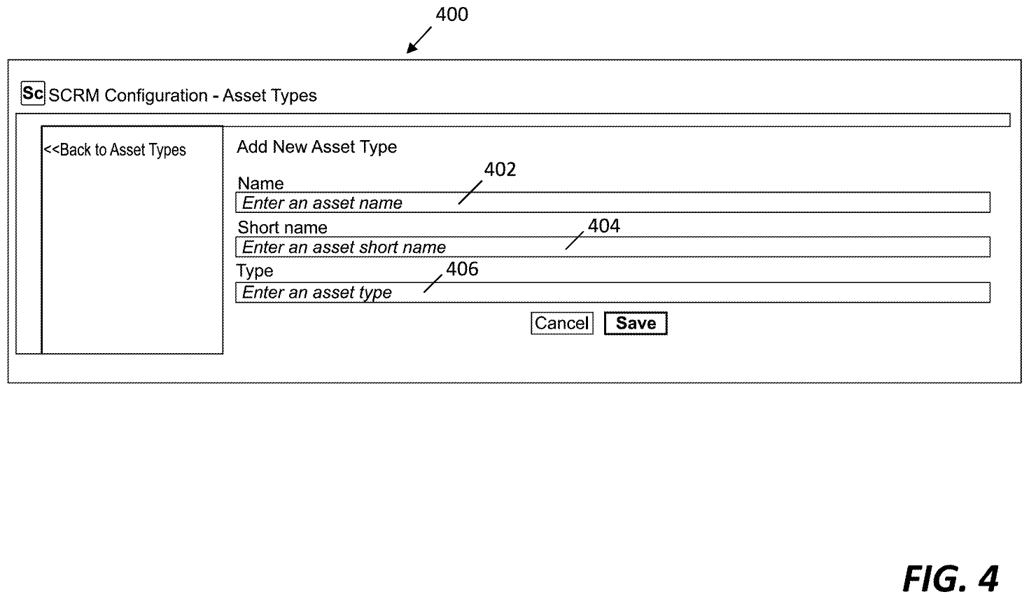

FIG. 4 shows an example of a user interface 400 generated by the configurator 200 for creating a new medical device asset in accordance with operation 302 of the method 300. The user interface 400 includes a first data field 402 to enter a name for the medical device asset, a second data field 404 to enter an abbreviated or shortened name for the medical device asset, and a third data field to enter an asset type for the medical device asset such as any one of the first, second, or third medical device asset types 122-126 that are described above.

In alternative examples, operation 302 can include editing a name for an existing medical device asset, optionally editing an abbreviated or shortened name for the existing medical device asset, and/or editing an asset type for the existing medical device asset. Operation 302 can further include changing and/or updating an image of the existing medical device asset.

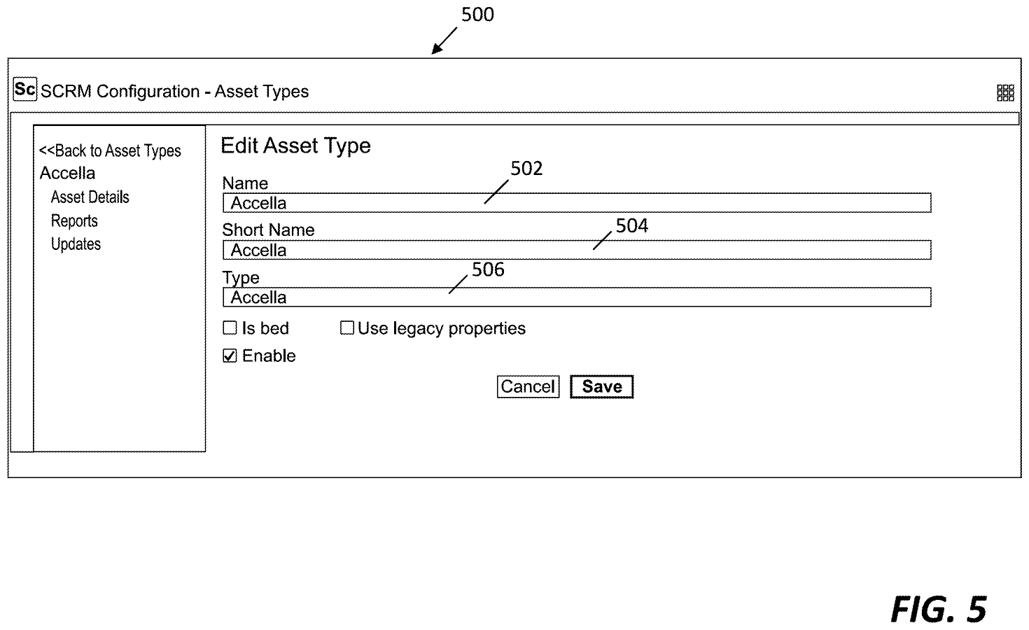

FIG. 5 shows another example of a user interface 500 generated by the configurator 200 for editing an existing medical device asset in accordance with operation 302 of the method 300. The user interface 500 includes a first data field 502 to edit the name for the existing medical device asset, a second data field 404 to edit the abbreviated or shortened name for the existing medical device asset, and a third data field to edit the asset type for the existing medical device asset such as to any one of the first, second, or third medical device asset types 122-126.

Referring back to FIG. 3, the method 300 includes an operation 304 of displaying a plurality of selectors for enabling device component information and functionalities for the new or existing medical device asset in the remote management system 100. Operation 304 can include displaying a first plurality of selectors for enabling device component information for the new or existing medical device asset in the remote management system 100.

As described above, the medical device assets may vary greatly between the plurality of medical device asset types 120, or even within the same medical device asset type, such that each medical device asset sends distinct kinds of service asset component information. The first plurality of selectors can be selected to enable or disable the component information in the remote management system 100 for new and existing medical device assets, as desired, and without requiring software coding or modifications to the remote management system 100. Thus, the configurator 200 improves the functioning of the remote management system 100 by eliminating the need for modifying the software code of the remote management system 100 to enable new or existing service asset component information in the remote management system 100 for a plurality of different medical device assets. The configurator 200 saves resources including labor and time that would otherwise need to be dedicated to modifying the software code of the remote management system 100 and can thus improve the extensibility and/or scalability of the remote management system 100 to new medical device assets.

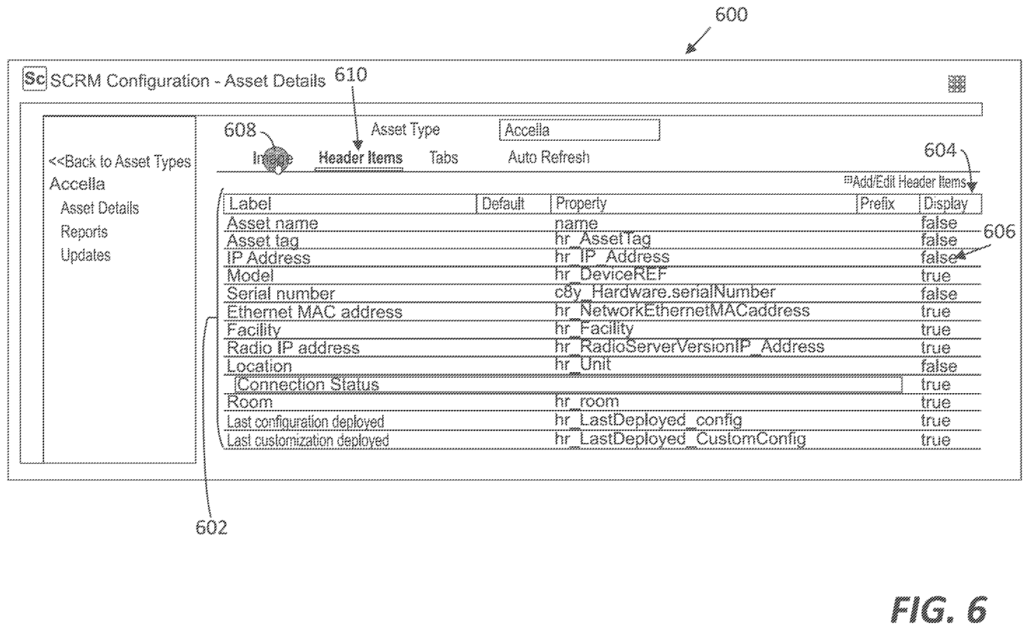

FIG. 6 shows an example of a user interface 600 generated by the configurator 200 that displays the first plurality of selectors for a new or existing medical device asset in accordance with operation 304. The device component information is listed in rows 602 under header items 610. The user interface 600 includes a column 604 that includes the first plurality of selectors 606 allowing a user to select whether to enable or disable a device component information listed in the rows 602 for the new or existing medical device asset in the remote management system 100. In this illustrative example, the user can enter/select “true” to enable the device component information, and can enter/select “false” to disable the device component information. Additional types of selectors for enabling or disabling the device component information are possible such that user interface 600 is provided by way of illustrative example.

The device component information listed in the rows 602 can include, without limitation, medical device asset name; medical device asset tag; medical device IP address; medical device model number; medical device serial number; medical device Ethernet MAC address; facility where medical device is located; medical device radio IP address; medical device geographic location; medical device connection status; room location of medical device; last configuration deployed; and last customization deployed. The user interface 600 also includes an image icon 608 for the user to add or edit an image of the medical device asset.

Referring back to FIG. 3, operation 304 can further include displaying a second plurality of selectors for enabling functionalities in the remote management system 100 for the new or existing medical device asset. As described above, the medical device assets may vary greatly between the plurality of medical device asset types 120, or even within the same medical device asset type, such that each medical device asset supports distinctive features within the remote management system 100. For example, one type of medical device asset may support log file request and retrieval, whereas another type of medical device asset may not support log file request and retrieval. The second plurality of selectors can be selected to enable or disable functionalities in the remote management system 100 for the new or existing medical device asset, as desired, and without requiring software coding or modifications to the remote management system 100. Thus, the configurator 200 further improves the functioning of the remote management system 100 by eliminating the need for modifying the software code of the remote management system 100 to enable new or existing functionalities within the remote management system 100 for a plurality of different medical device assets. The configurator 200 saves resources including labor and time that would otherwise need to be dedicated to modifying the software code of the remote management system 100, and can thus improve the extensibility and/or scalability of the remote management system 100 to new medical device assets.

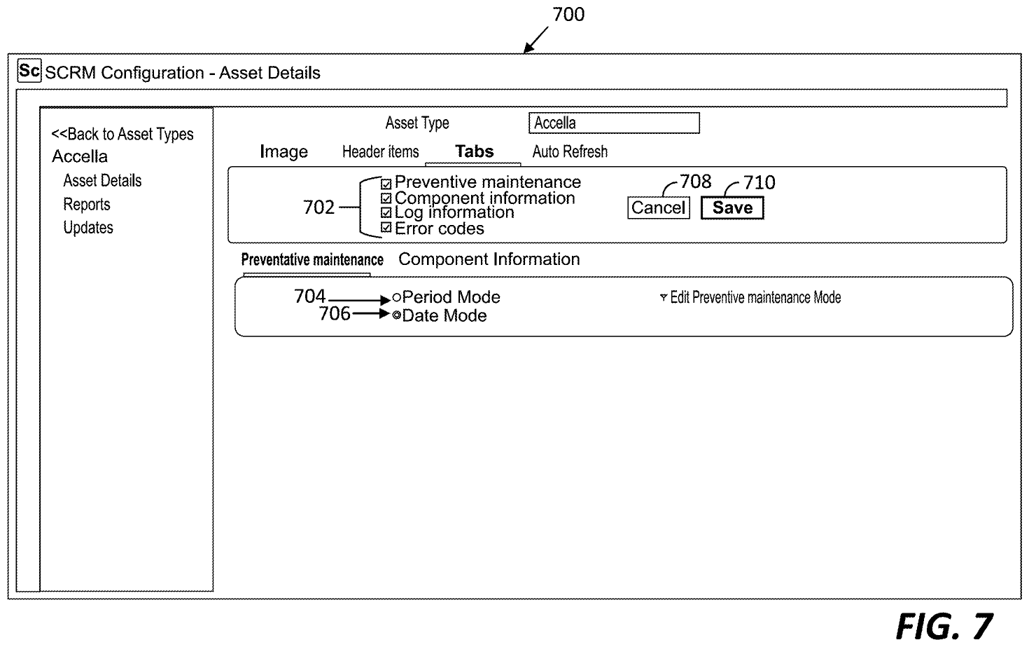

FIG. 7 shows an example of a user interface 700 generated by the configurator 200 that displays the second plurality of selectors 702 for the new or existing medical device asset in accordance with operation 304. The second plurality of selectors 702 can be selected to enable or disable preventative maintenance tracking, component information tracking, log request and retrieval, and error code functionalities in the remote management system 100 for the new or existing medical device asset. Additional functionalities can be included the second plurality of selectors 702 such that these functionalities are provided by way of illustrative example. For example, the second plurality of selectors 702 can further be selected to configure access point location tracking, firmware updates, and configuration updates functionalities in the remote management system 100 for the new or existing medical device asset.

As further shown in FIG. 7, the user interface 700 allows the user to configure the preventative maintenance function in the remote management system 100 for a new or existing medical device asset. For example, the user can select a first icon 704 to configure the preventative maintenance function in the remote management system 100 to be in a period mode (i.e., the preventative maintenance occurs during predefined periods) and can include a second icon 706 to configure the preventative maintenance function in the remote management system 100 to be in a date mode (i.e., the preventative maintenance occurs during predefined dates).

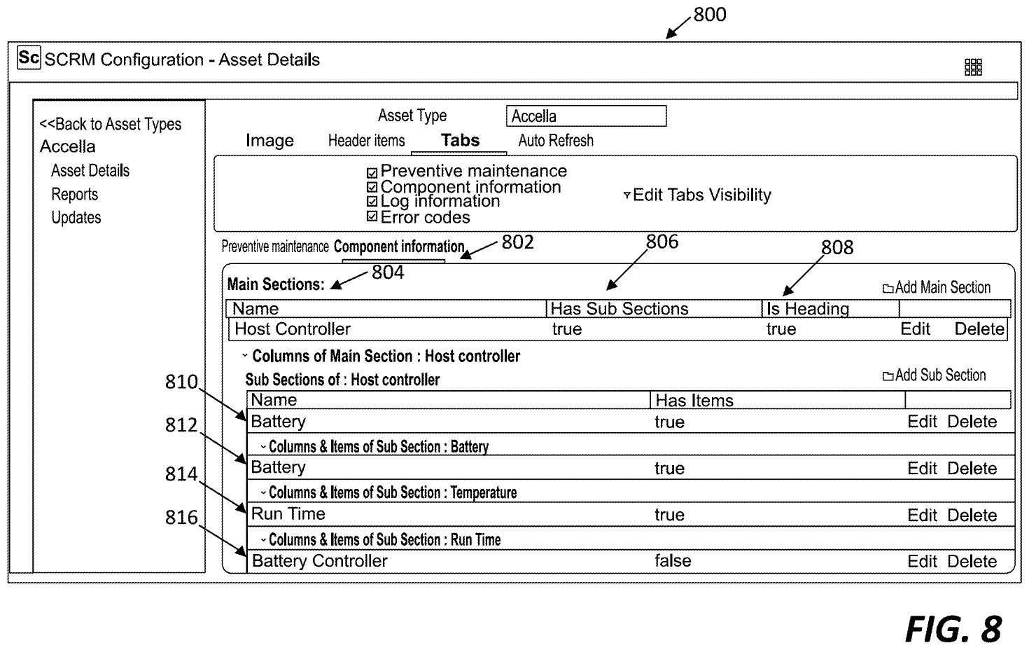

FIG. 8 shows a user interface 800 generated by the configurator 200 that illustrates in more detail configuring the component information details and tracking based on a selection of the second plurality of selectors 702 included in the user interface of FIG. 7. The user interface 800 displays component information 802 having a main section 804 that lists components of the medical device asset for selection. In this example, a host controller is selected under the main section 804. Selectors (e.g., “true” or “false”) are displayed for selecting one or more options for the component information such as a first option 806 of whether the component information includes sub sections and a second option 808 of whether the component information is displayed in a heading of the medical device asset in the remote management system 100.

As further shown in FIG. 8, the sub sections 810-816 are displayed under the main section 804. For example, a first sub section 810 is displayed for the battery of the host controller, a second sub section 812 is displayed for the temperature of the host controller, a third sub section 814 is displayed for the run time of the host controller, and a fourth sub section 816 is displayed for the battery controller of the host controller. The sub sections 810-816 each include selectors (e.g., “true” or “false”) for selecting whether to display and/or track the parameters of the component (e.g., host controller) in the main section 804.

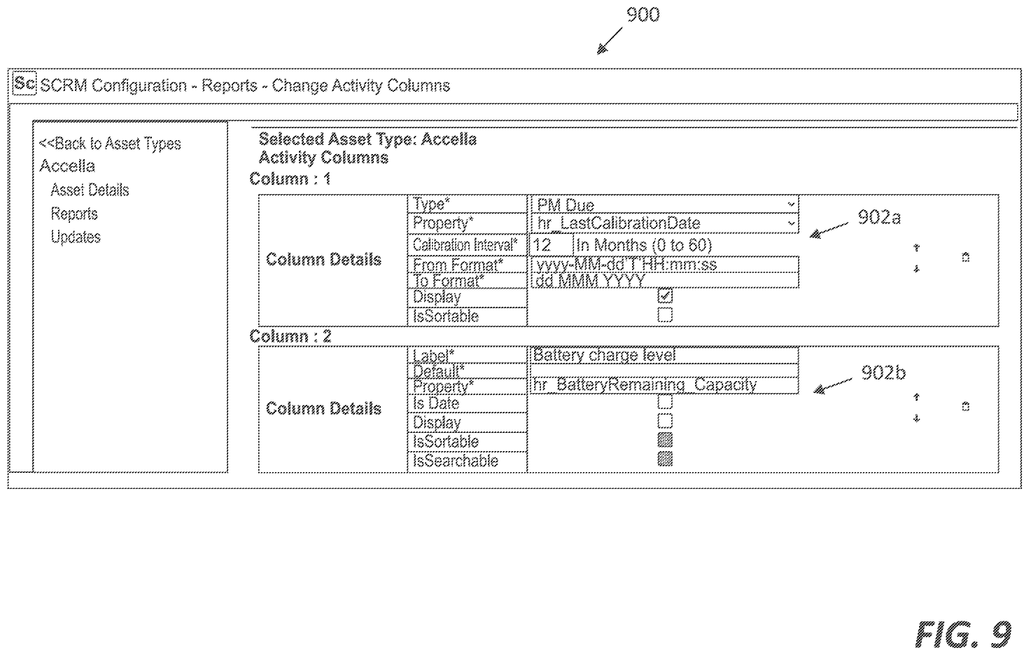

FIG. 9 shows an example of a user interface 900 generated by the configurator 200 that includes a third plurality of selectors 902a, 902b for customizing a report type in the remote management system 100 for a new or existing medical device asset. In this example, the third plurality of selectors 902a, 902b are selectable for selecting which fields to display for each column of a report type (e.g., an activity report) enabled in the remote management system 100 for a medical device asset such as an Accella® Smart Bed 126a.

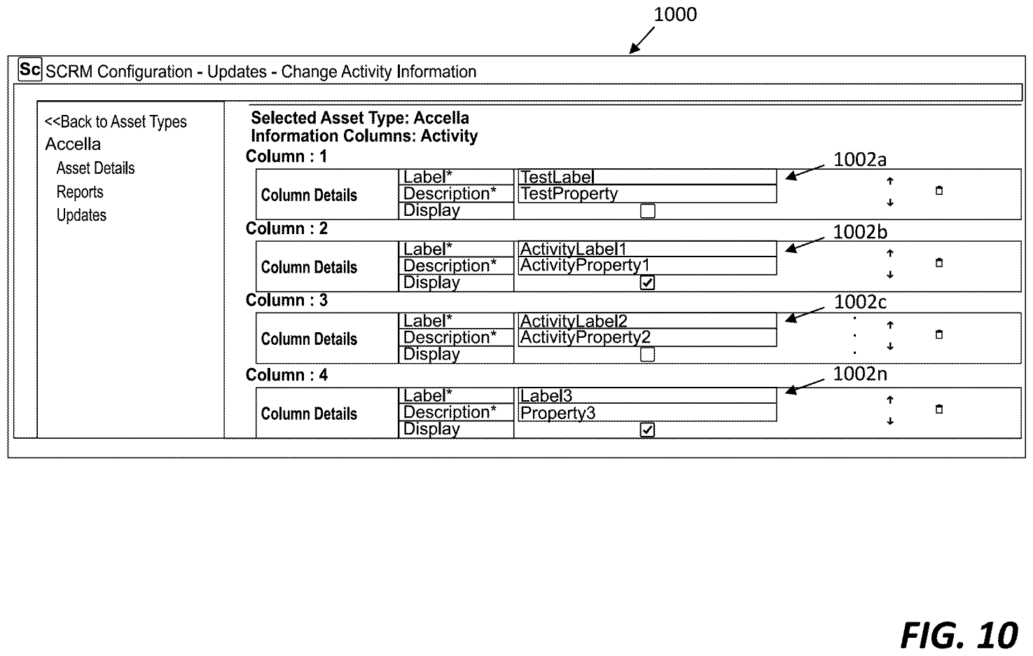

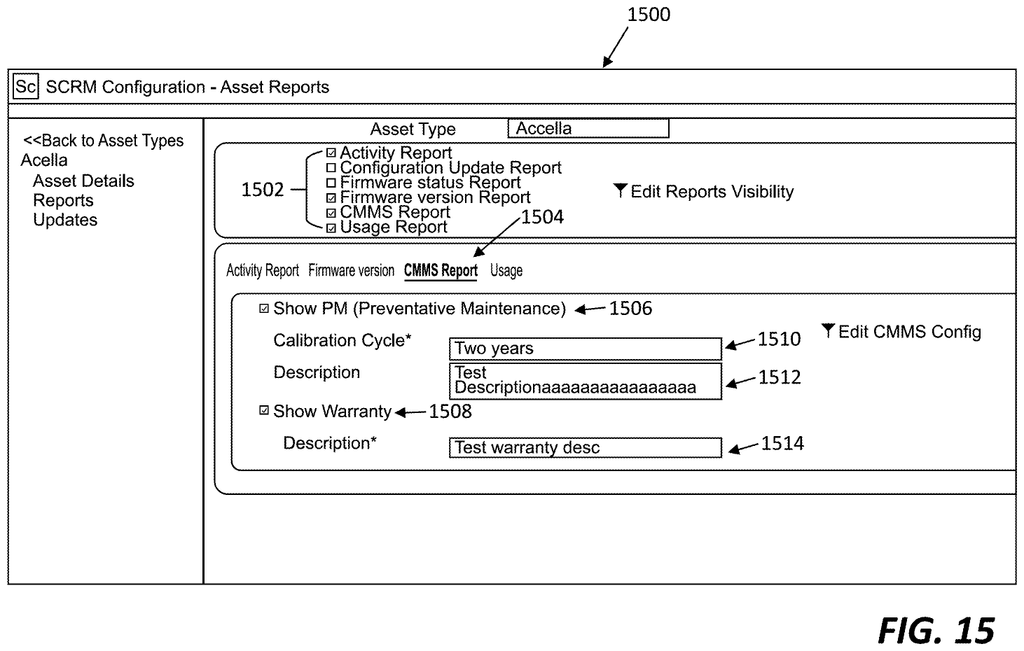

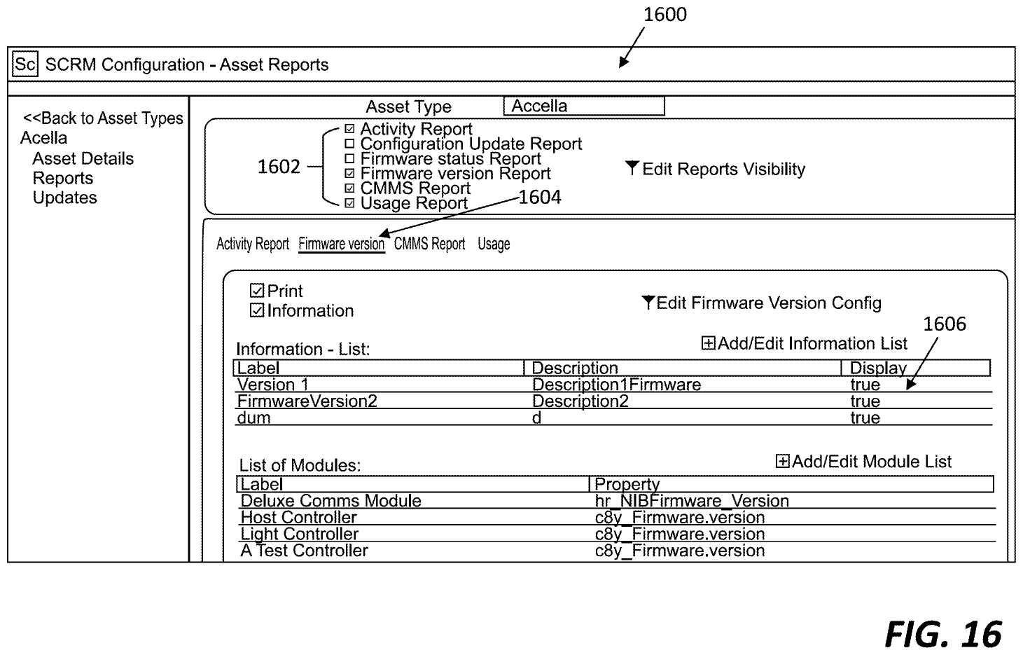

FIG. 10 shows another example of a user interface 1000 generated by the configurator 200 that includes a third plurality of selectors 1002a-1002n for configuring each column of a report type (e.g., an activity report) in the remote management system 100 for a new or existing medical device asset. In some examples, the one or more report types include canned reports for remote management of the medical device asset. Additional examples of how the configurator 200 can be used to customize the reports in the remote management system 100 for the medical device assets are shown in FIGS. 15 and 16, which will be described in more detail below.

Referring back to FIG. 3, the method 300 next includes an operation 306 of receiving one or more selections of the first and second pluralities of selectors for the medical device asset. Examples of the first and second pluralities of selectors are shown in FIGS. 6-10.

In some instances, the one or more selections of the first and second pluralities of selectors are received in operation 306 following a selection of a save icon 708 (see FIG. 7). In further examples, the one or more selections of the first and second pluralities of selectors are discarded following selection of a cancel icon 710 (see FIG. 7).

The method 300 includes an operation 308 of configuring the remote management system 100 to remotely manage the medical device asset based on the device component information and the functionalities selected for the new medical device asset type. The one or more selections of the device component information and the functionalities configure the remote management system 100 to communicate with the internet of things network 110 for remote management of the medical device asset. Also, one or more selections of the first and second pluralities of selectors can configure the remote management system 100 to enable a third-party user to interface with the medical device asset type. As used herein, the third-party user is not the medical device manufacturer or the customer of the medical device asset.

The method 300 improves the functioning of the remote management system 100 by eliminating the need for modifying the software code of the remote management system 100 to enable new or existing service asset component information and functionalities within the remote management system 100 for a plurality of different medical device assets. The configurator 200 saves resources including labor and time that would otherwise need to be dedicated to modifying the software code of the remote management system 100 and can thus significantly improve the extensibility and/or scalability of the remote management system 100.

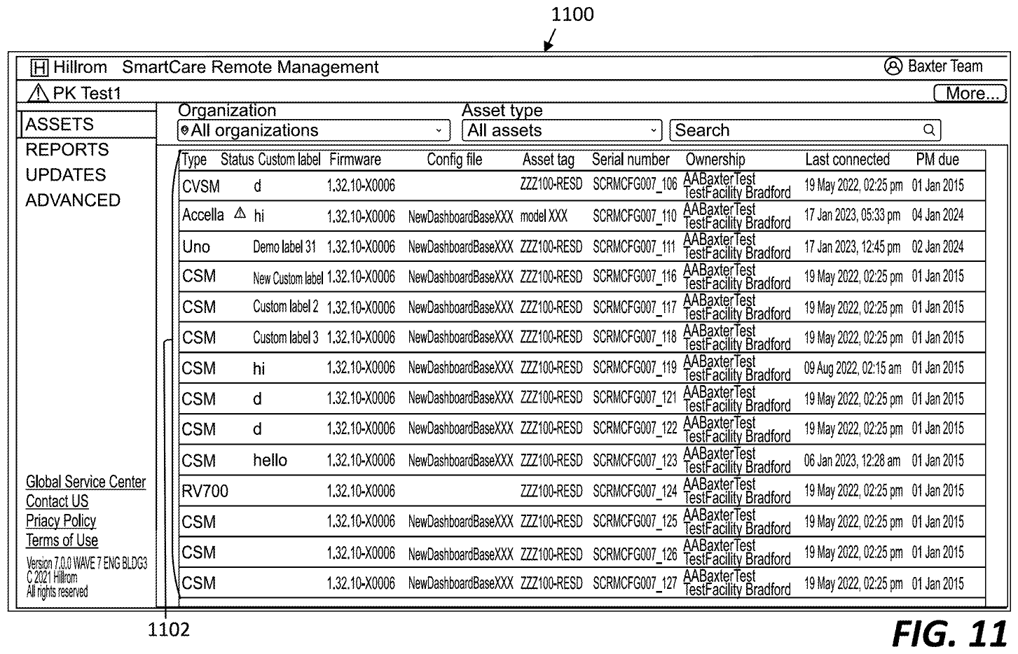

FIG. 11 shows an example of a user interface 1100 displayed by the remote management system 100 once it has been configured by the configurator 200 in accordance with the operations of the method 300. The user interface 1100 displays a list 1102 of medical device assets. In this example, a cursor is shown over an “Accella” medical device asset.

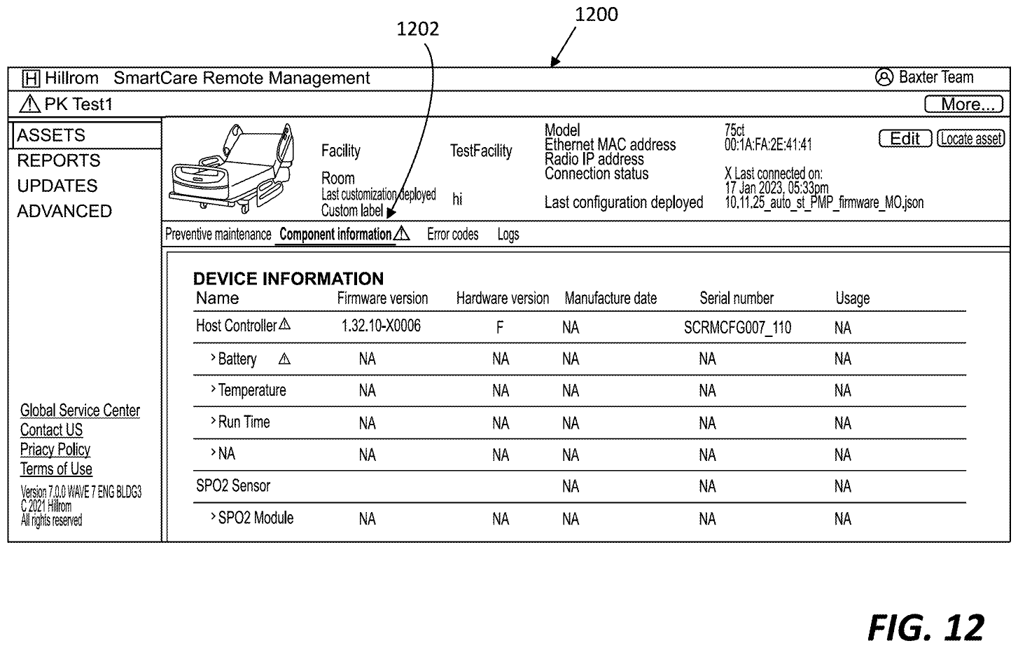

FIG. 12 shows an example of a user interface 1200 displayed by the remote management system 100 after selection of the “Accella” medical device asset shown in the user interface 1100 of FIG. 11. As shown in FIG. 12, the user interface 1200 includes a component information display 1202 based on the selections from the user interface 800 of FIG. 8. For example, the component information display 1202 includes various pieces of information for the host controller component such as the battery, temperature, and run time. Additional components of the Accella medical device asset can be included in the component information display 1202 based on the selections of the first plurality of selectors such as one or more sensors that can be incorporated into the Accella medical device asset including an SpO2 sensor.

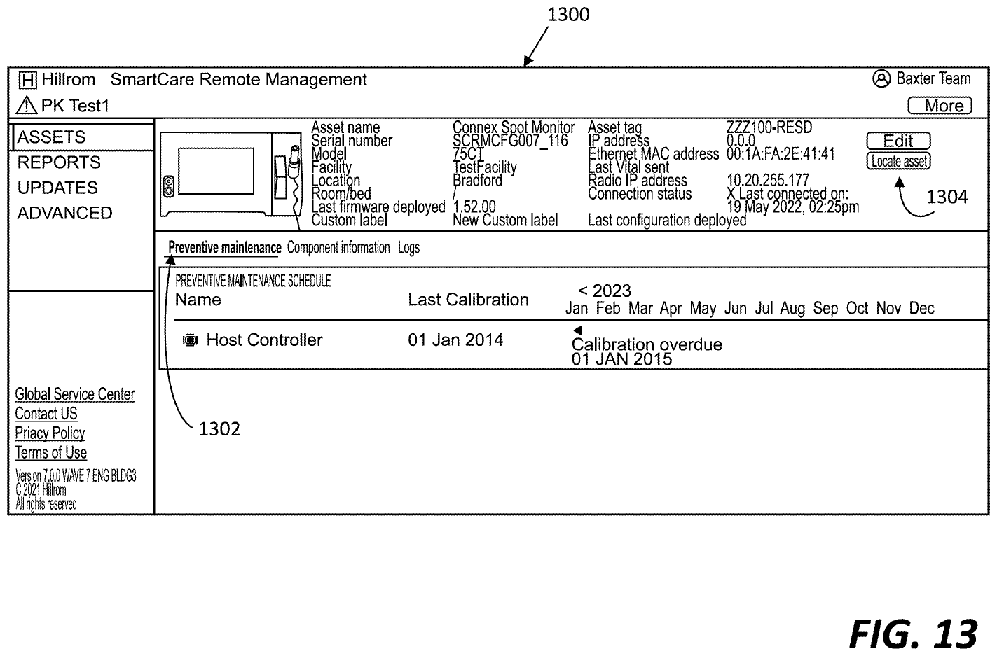

FIG. 13 shows another example of a user interface 1300 displayed by the remote management system 100 after a selection of a Connex® Spot Monitor (CSM) medical device asset shown in the user interface 1100 of FIG. 11. The user interface 1300 includes a preventive maintenance report 1302 based on one or more selections of the third plurality of selectors 902a in the user interface 900 of FIG. 9. For example, the preventive maintenance report 1302 can include a preventive maintenance schedule for a component of the medical device asset such as the host controller, and can include data such as when the component was last calibrated.

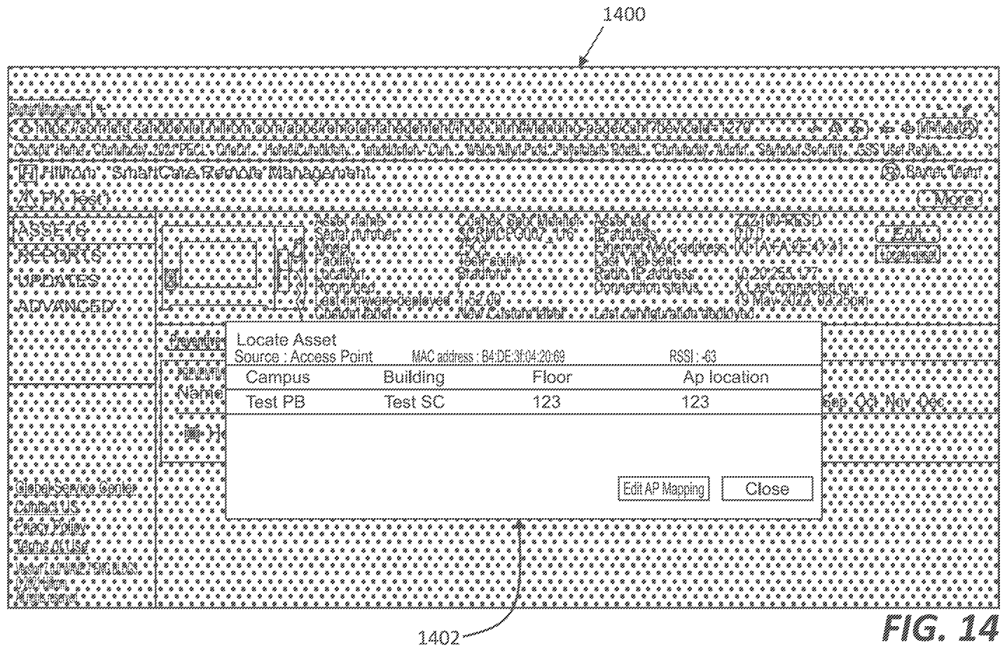

FIG. 14 shows another example of a user interface 1400 displayed by the remote management system 100 after selection of a locate asset icon 1304 included in the user interface 1300 of FIG. 13. The user interface 1400 includes an access point location tracking 1402 that can be enabled for the medical device asset based on selection of the one or more first plurality of selectors. The access point location tracking 1402 identifies the location of the medical device asset (e.g., campus, building, floor, room number) by detecting a location where the MAC address of the medical device asset connects to the internet of things network 110.

FIG. 15 shows another example of a user interface 1500 generated on the configurator 200 for configuring the remote management system 100. The user interface 1500 includes a third plurality of selectors 1502 for configuring one or more report types in the remote management system 100 for a new or existing medical device asset. For example, the third plurality of selectors 1502 are selectable for configuring one or more of an activity report, a configuration update report, a firmware status report, a firmware version report, a computerized maintenance management system (CMMS) report, a usage report, and other similar report types.

In the example shown in FIG. 15, a CMMS report tab 1504 is selected for customizing a CMMS report for the medical device asset. For example, the third plurality of selectors 1502 include selectors 1506, 1508 that are selectable for selecting which fields to display in the CMMS report when enabled in the remote management system 100 for the medical device asset. In FIG. 15, both selectors 1506, 1508 are selected for including preventive maintenance and warranty fields in the CMMS report, respectively. Under the selector 1506, additional options are provided such as a first option 1510 to set the calibration cycle, and a second option 1512 to provide a description. Similarly, under the selector 1506, a third option 1514 is provided to provide a description of the warranty. FIG. 15 is provided by way of illustrative example, and it is contemplated that different formats and selectors may be provided for customizing a report for the medical device asset in the remote management system 100.

FIG. 16 shows another example of a user interface 1600 generated on the configurator 200 for configuring the remote management system 100. The user interface 1600 includes a third plurality of selectors 1602 for configuring one or more report types in the remote management system 100 for a new or existing medical device asset. In the example shown in FIG. 16, a firmware version report tab 1604 is selected for customizing a firmware version report for the medical device asset. In this example, the third plurality of selectors 1602 include selectors 1606 that are selectable for selecting which fields to display in the firmware version report when enabled in the remote management system 100 for the medical device asset.

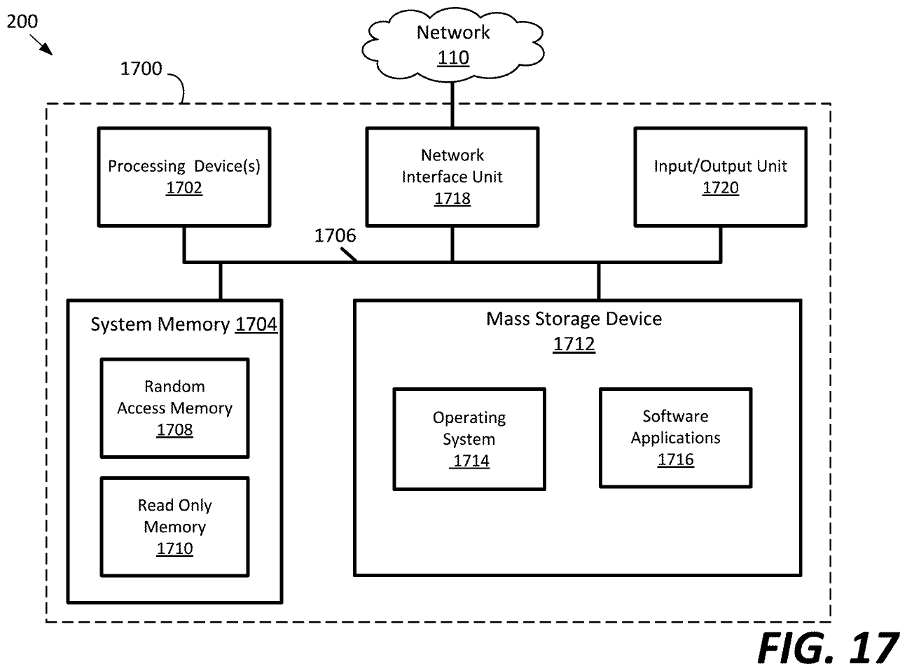

FIG. 17 schematically illustrates an example of computing hardware 1700 of the configurator 200 for implementing the aspects of the present disclosure. The remote management system 100 and the medical device assets can include similar computing hardware.

As shown in the example provided in FIG. 17, the computing hardware 1700 includes at least one processing device 1702, a system memory 1704, and a system bus 1706 that couples the system memory 1704 to the at least one processing device 1702. Examples of the at least one processing device 1702 can include central processing units (CPU).

The system memory 1704 includes a random-access memory (“RAM”) 1708 and a read-only memory (“ROM”) 1710. Basic input and output logic routines to transfer information between elements within the configurator 200, such as during startup, can be stored in the ROM 1710. The system memory 1704 is an example of a computer-readable memory device.

The configurator 200 can further include a mass storage device 1712 that can include an operating system 1714, and that can store one or more software applications 1716. The mass storage device 1712 is connected to the at least one processing device 1702 through the system bus 1706. The mass storage device 1712 and associated computer-readable data storage media provide non-volatile, non-transitory storage for the configurator 200.

Although the description of computer-readable data storage media contained herein refers to the mass storage device 1712, it should be appreciated by those skilled in the art that computer-readable data storage media can be any available non-transitory, physical device or article of manufacture from which the configurator 200 can read data and/or instructions. The computer-readable storage media can be comprised of entirely non-transitory media. The mass storage device 1712 is another example of a computer-readable memory device.

Computer-readable data storage media include volatile and non-volatile, removable, and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The configurator 200 operates in a networked environment using logical connections to the other devices through the internet of things network 110. The configurator 200 connects to the internet of things network 110 through a network interface unit 1718 connected to the system bus 1706. The network interface unit 1718 can also connect to additional types of communications networks and devices, including Bluetooth, Wi-Fi, and cellular telecommunications networks including 4G and 5G networks. The network interface unit 1718 can connect the configurator 200 to additional networks, systems, and devices. The configurator 200 further includes an input/output unit 1720 for receiving and processing inputs and outputs.

The mass storage device 1712 and the RAM 1708 can store software instructions and data. The software instructions can include an operating system 1714 for operating the configurator 200. The mass storage device 1712 and/or the RAM 1708 can also store the software applications 1716, which when executed by the at least one processing device 1702, provide the various functions and aspects of the configurator 200 discussed herein.

The various embodiments described above are provided by way of illustration only and should not be construed to be limiting in any way. Various modifications can be made to the embodiments described above without departing from the true spirit and scope of the disclosure.

What is claimed is:

1. An apparatus for configuring a remote management system that is used for remotely managing a plurality of medical device assets, the apparatus comprising:
at least one processing device; and
a memory device storing instructions which, when executed by the at least one processing device, cause the at least one processing device to:
display a user interface including:
a first plurality of selectors for enabling device component information in the remote management system; and
a second plurality of selectors for enabling functionalities in the remote management system;
receive from the user interface one or more selections of the first and second pluralities of selectors for a medical device asset; and
configure the remote management system to remotely manage the medical device asset based on the one or more selections of the first and second pluralities of selectors for the medical device asset.

2. The apparatus of claim 1, wherein the medical device asset is a patient support system, a vital signs monitoring device, or an eye imaging device.

3. The apparatus of claim 1, wherein the one or more selections of the device component information and the functionalities configure the remote management system to communicate with an internet of things network for remote management of the medical device asset.

4. The apparatus of claim 1, wherein the instructions, when executed by the at least one processing device, further cause the at least one processing device to:
configure the remote management system to enable a third-party user to interface with the medical device asset.

5. The apparatus of claim 1, wherein the first plurality of selectors for the device component information enables configuration in the remote management system of the device component information for the medical device asset, the device component information including data relevant to one or more components of the medical device asset.

6. The apparatus of claim 1, wherein the second plurality of selectors for the functionalities are selectable to enable in the remote management system functions including at least one of log file request and retrieval, preventative maintenance tracking, access point location tracking, firmware updates, and configuration updates for the medical device asset.

7. The apparatus of claim 1, wherein the user interface further includes a third plurality of selectors for customizing reports in the remote management system for the medical device asset.

8. A method of configuring a remote management system that remotely manages a plurality of medical device assets, the method comprising:
displaying a user interface including:
a first plurality of selectors for enabling device component information in the remote management system; and
a second plurality of selectors for enabling functionalities in the remote management system;
receiving from the user interface one or more selections of the first and second pluralities of selectors for a medical device asset; and
configuring the remote management system to remotely manage the medical device asset based on the one or more selections of the first and second pluralities of selectors for the medical device asset.

9. The method of claim 8, wherein the medical device asset is a patient support system, a vital signs monitoring device, or an eye imaging device.

10. The method of claim 8, further comprising:
configuring the remote management system to communicate with an internet of things network for remote management of the medical device asset based on the one or more selections of the device component information and the functionalities.

11. The method of claim 8, further comprising:
configuring the remote management system to enable a third-party user to interface with the medical device asset.

12. The method of claim 8, further comprising:
configuring device component information including data relevant to at least one component of the medical device asset based on one or more selections of the first plurality of selectors.

13. The method of claim 8, further comprising:
configuring preventative maintenance information for the medical device asset in the remote management system based on a selection of the first plurality of selectors.

14. The method of claim 8, further comprising:
enabling in the remote management system functionalities for the medical device asset based on one or more selections of the second plurality of selectors, the functionalities including one or more of log file request and retrieval, preventative maintenance tracking, access point location tracking, firmware updates, and configuration updates.

15. The method of claim 8, further comprising:
providing a third plurality of selectors in the user interface, the third plurality of selectors selectable for customizing reports for the medical device asset in the remote management system.

16. A non-transitory computer-readable data storage medium comprising instructions that, when executed, cause at least one computing device to:
display a user interface including:
a first plurality of selectors for enabling device component information in a remote management system; and
a second plurality of selectors for enabling functionalities in the remote management system;
receive from the user interface selections of the first and second pluralities of selectors for a medical device asset; and
configure the remote management system to remotely manage the medical device asset based on the selections of the first and second pluralities of selectors for a medical device asset.

17. The non-transitory computer-readable data storage medium of claim 16, wherein the medical device asset is a patient support system, a vital signs monitoring device, or an eye imaging device.

18. The non-transitory computer-readable data storage medium of claim 16, wherein the instructions, when executed, further cause the at least one computing device to:
configure the remote management system to communicate with an internet of things network for remote management of the medical device asset based on the one or more selections of the device component information and the functionalities.

19. The non-transitory computer-readable data storage medium of claim 16, wherein the instructions, when executed, further cause the at least one computing device to:
configure the remote management system to enable a third-party user to interface with the medical device asset.

20. The non-transitory computer-readable data storage medium of claim 16, wherein the instructions, when executed, further cause the at least one computing device to:
configure the device component information relevant to at least one component of the medical device asset based on one or more selections of the first plurality of selectors;
configure preventative maintenance for the medical device asset in the remote management system based on one or more selections of the first plurality of selectors; and
enable in the remote management system functionalities for the medical device asset based on one or more selections of the second plurality of selectors, the functionalities including one or more of log file request and retrieval, preventative maintenance tracking, access point location tracking, firmware updates, and configuration updates.
